(19) United States
(12) Patent Application Publication
(10) Pub. No.: US 2026/0083412 A1
(43) Pub. Date: Mar. 26, 2026

(54) RADIOTHERAPY BED

(71) Applicant: HITACHI HIGH-TECH CORPORATION, Tokyo (JP)

(72) Inventors: Masayuki ARAKAWA, Tokyo (JP); Yu SAITOH, Tokyo (JP); Takashi ISHIZUKA, Tokyo (JP)

(21) Appl. No.: 19/112,306

(22) PCT Filed: Jul. 19, 2023
(86) PCT No.: PCT/JP2023/026419

§ 371 Date: Mar. 17, 2025

(30) Foreign Application Priority Data
Oct. 17, 2022 (JP) 2022-166466

(51) Int. Cl. A61B 6/04; A61N 5/10

(52) U.S. Cl. CPC A61B 6/0407; A61N 5/1077

(57) ABSTRACT

The radiotherapy bed includes a base member, a first bed member that is placed on the base member and whose position with respect to the base member can be mechanically controlled, and a second bed member that is placed on the first bed member and whose position with respect to the first bed member can be mechanically controlled. The second bed member is movable in a direction in which a treatment subject placed on the second bed member is carried into an area where a radiation is to be emitted by a radiotherapy device and in a direction in which the treatment subject is carried out from the area where the radiation is to be emitted by the radiotherapy device, and the first bed member has a grip portion on a surface along a carrying-in direction of the treatment subject.

### TECHNICAL FIELD

The present invention relates to a radiotherapy bed.

### BACKGROUND ART

In the related art, JP2010-537781A (PTL 1) describes a technique of a device for carrying and moving a patient. This publication discloses that “A patient supporting device for use in a medical facility is provided. The patient support device includes a base and a table assembly coupled to the base. The table assembly includes a lower support portion and an upper support portion that is coupled to the lower support portion and movable with respect to the lower support portion. At least one of the upper support portion and the lower support portion includes a bearing layer capable of improving performance of the patient supporting device when the upper support portion moves with respect to the lower support portion.”

### CITATION LIST

### Patent Literature

PTL 1: JP2010-537781A

### SUMMARY OF INVENTION

### Technical Problem

In radiotherapy, it is necessary to position a patient with respect to a radiation emitting unit with high accuracy. Therefore, a mechanism capable of mechanically controlling a horizontal position, a vertical position, an inclination, and the like is mounted on a radiotherapy bed for carrying a patient who is a treatment subject.

In the radiotherapy bed, it is very important to prevent a failure or an erroneous operation. This is because the repair cost is high and the time until the completion of the repair significantly affects therapeutic planning.

One of the causes of a failure or an erroneous operation of the radiotherapy bed is manual position control. Specifically, this is a case where an operator who operates the radiotherapy bed manually moves the bed to perform rough positioning and then performs positioning by precise control with a machine. Manual position control is not recommended and the operator does not know which part of the bed is to be held and moved, and therefore, the operation causes a failure or an erroneous operation.

Another cause of the failure or the erroneous operation is accidental contact of the treatment subject with the operation unit. Similarly, direct contact of the operation unit with vomit or body fluid of the treatment subject may cause a failure or an erroneous operation.

Therefore, an object of the invention is to prevent a failure or a malfunction by preventing inappropriate contact with a radiotherapy bed.

### Solution to Problem

In order to achieve the above object, a typical radiotherapy bed according to one aspect of the invention includes: a base member; a first bed member that is placed on the base member and whose position with respect to the base member is mechanically controllable; and a second bed member that is placed on the first bed member and whose position with respect to the first bed member is mechanically controllable. The second bed member is movable in a direction in which a treatment subject placed on the second bed member is carried into an area where a radiation is to be emitted by a radiotherapy device and in a direction in which the treatment subject is carried out from the area where the radiation is to be emitted by the radiotherapy device, and the first bed member has a grip portion on a surface along a carrying-in direction of the treatment subject.

A typical radiotherapy bed according to one aspect of the invention includes: a base member; and a bed member that is placed on the base member and whose position with respect to the base member is mechanically controllable. The bed member allows a positional relationship between a treatment subject placed on the bed member and an area where a radiation is to be emitted by a radiotherapy device to be controllable, and the bed member has a grip portion on a surface along a height direction of the treatment subject.

### Advantageous Effects of Invention

According to the invention, inappropriate contact with the radiotherapy bed can be prevented. Problems, configurations, and effects other than those described above will become apparent by the following description of embodiments.

### BRIEF DESCRIPTION OF DRAWINGS

FIG. 1 is a view illustrating four sides of a radiotherapy bed.

FIG. 2 is a perspective view of the radiotherapy bed.

FIG. 3 is a view illustrating a first bed member.

FIG. 4 is a cross-sectional view of the radiotherapy bed.

FIG. 5 is a view illustrating a positional relationship among a grip portion, an operation unit, and an erroneous operation prevention button.

FIG. 6 is a view illustrating operations.

### DESCRIPTION OF EMBODIMENTS

Hereinafter, an embodiment will be described with reference to the drawings.

### Embodiment 1

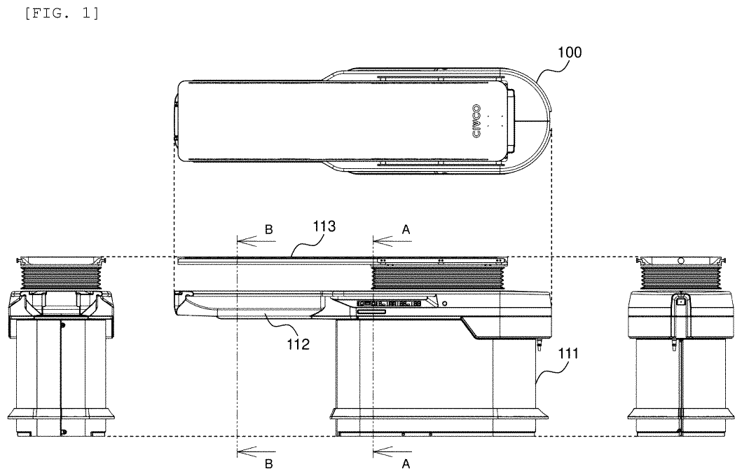

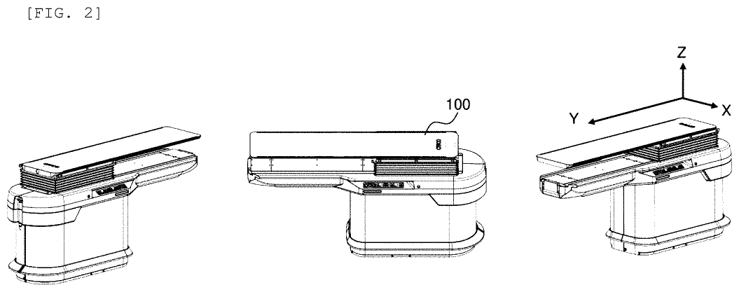

FIG. 1 is a view illustrating four sides of a radiotherapy bed, and FIG. 2 is a perspective view of the radiotherapy bed. A radiotherapy bed 100 includes a base member 111, a first bed member 112, and a second bed member 113.

The base member 111 is provided in contact with a floor.

The first bed member 112 is placed on the base member 111, and a position of the first bed member 112 with respect to the base member 111 can be mechanically controlled.

The second bed member 113 is placed on the first bed member 112, and a position of the second bed member 113 with respect to the first bed member 112 can be mechanically controlled.

Specifically, the second bed member 113 is movable in a direction (Y) in which a treatment subject placed on the second bed member 113 is carried into a radiotherapy device and in a direction in which the treatment subject is carried out from an inside of the radiotherapy device. Further, the second bed member 113 can adjust an angle of a direction (roll) of rotation with respect to a carrying-in direction and an angle of an inclination (pitch) with respect to the carrying-in direction.

The inside of the radiotherapy device is an area where a radiation is to be emitted by a radiation emitting unit, and is, for example, an inside of a gantry.

The second bed member 113 is long in the carrying-in direction, and the treatment subject is placed with the head facing the radiotherapy device. That is, the carrying-in direction coincides with a height direction of the treatment subject.

The first bed member 112 is movable in a horizontal direction (X) and a vertical direction (Z) with respect to the floor. Here, a movement of the first bed member 112 in the horizontal direction is a movement of the second bed member 113 in a direction perpendicular to the carrying-in direction. Similarly, a movement of the first bed member 112 in the vertical direction is a movement in a direction perpendicular to the carrying-in direction of the second bed member 113.

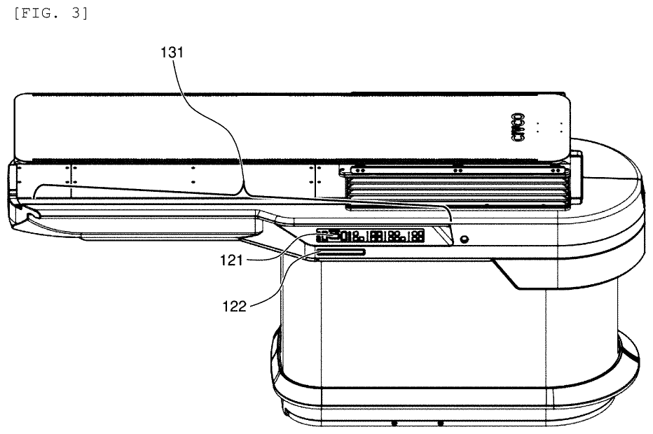

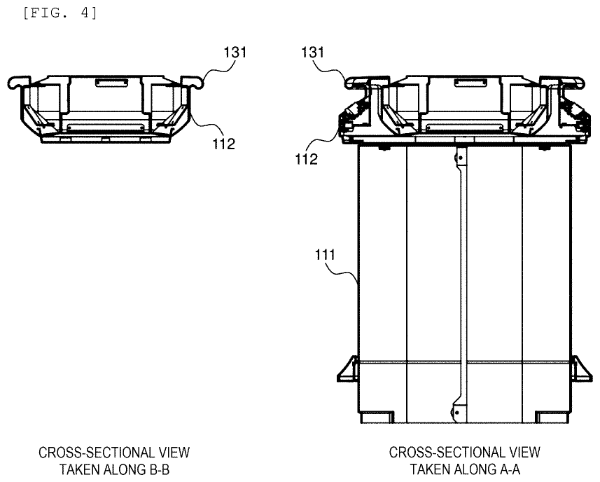

FIG. 3 is a view illustrating the first bed member 112. FIG. 4 is a cross-sectional view of the radiotherapy bed 100.

As illustrated in FIG. 3, the first bed member 112 includes a grip portion 131, an operation unit 121, and an erroneous operation prevention button 122. The grip portion 131 is provided on a surface along the carrying-in direction of the treatment subject. Specifically, as illustrated in FIG. 4, the grip portion 131 has a shape in which an upper surface of the first bed member 112 protrudes in the direction (X) perpendicular to the carrying-in direction (Y), and a recessed portion on which a finger of the operator is caught is provided below the protruding portion.

FIG. 4 illustrates cross-sectional views taken along lines A-A and B-B illustrated in FIG. 1. As described above, the grip portion 131 extends over a certain length on a side of the first bed member 112. The grip portion 131 is visible to the operator and has a shape including a recessed portion on a lower side, and therefore, the operator can easily recognize that the grip portion 131 is a place suitable for gripping. Therefore, when the operator intends to manually move the position of the bed in the horizontal direction (X) or the vertical direction (Y), the operator can be guided to grip the grip portion 131. In particular, due to a structure of the radiotherapy bed 100, there is a high possibility of causing a failure when a force is applied by putting a hand on the second bed member 113, and thus naturally guiding the operator to grip the grip portion 131 of the first bed member 112 is effective in preventing a failure. When the position of the bed is manually moved, it is assumed that the operator uses both hands. Extending the grip portion 131 in a certain length has an effect of guiding the positions of both hands. In other words, the “certain length” of the grip portion 131 is determined based on the positional relationship between both hands assumed when a heavy object is moved.

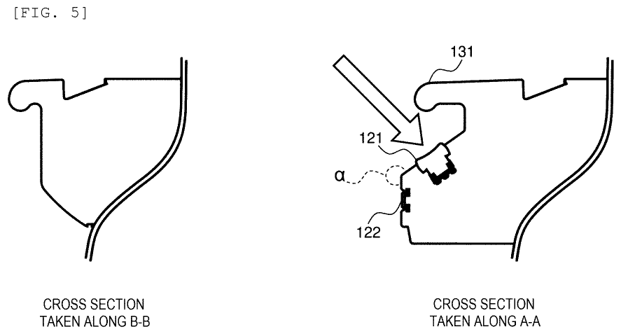

FIG. 5 is a view illustrating a positional relationship among the grip portion, the operation unit, and the erroneous operation prevention button. As illustrated in FIG. 5, the first bed member 112 includes a first predetermined angle surface having a first predetermined angle with respect to the upper surface of the first bed member 112.

The operation unit 121 is provided on the first predetermined angle surface. The operation unit 121 is provided at a position where the grip portion 131 serves as an eave. The operation unit 121 is disposed such that the eave does not reach an extension line in a direction perpendicular to the first predetermined angle surface from the operation unit 121.

Further, the first bed member 112 includes a second predetermined angle surface having a second predetermined angle different from the first predetermined angle with respect to the upper surface of the first bed member 112.

The erroneous operation prevention button 122 is provided on the second predetermined angle surface. The erroneous operation prevention button 122 and the operation unit 121 are provided close to each other, and the erroneous operation prevention button 122 and the operation unit 121 can be simultaneously operated with one hand.

The second predetermined angle surface may be perpendicular to the floor, but is not limited thereto.

An angle a between the second predetermined angle surface and the first predetermined angle surface is 180 degrees or more. The angle a of 180 degrees or more facilitates simultaneous operation with one hand and has an effect of preventing a situation in which the erroneous operation prevention button 122 and the operation unit 121 are simultaneously operated due to accidental contact, vomit, or the like.

The operation unit 121 is implemented by buttons, levers, and the like for receiving various operations on the radiotherapy bed 100. The operation unit 121 is preferably waterproofed. The operation received by the operation unit 121 includes the horizontal movement and the vertical movement of the first bed member 112, the movement and angle adjustment of the second bed member 113, and the like.

The operation unit 121 receives at least a part of operations on condition that the erroneous operation prevention button 122 is being pressed.

The angle of the first predetermined angle surface and the positional relationship of the grip portion 131 are defined so that the operation unit 121 is visible to the operator. On the other hand, the erroneous operation prevention button 122 does not need to be visible to the operator. The erroneous operation prevention button 122 is disposed in a sufficient size below the position of the operation unit 121 so as to be operable without visual inspection.

As illustrated in FIG. 5, when the grip portion 131 serves as an eave, the operation unit 121 can be prevented from being directly wetted with a liquid (for example, vomit or body fluid of a treatment subject). In addition, when the grip portion 131 serves as an eave, accidental contact with the operation unit 121 can be prevented even when the hand or foot of the treatment subject falls from the bed. Therefore, a failure or an erroneous operation of the operation unit 121 can be effectively prevented.

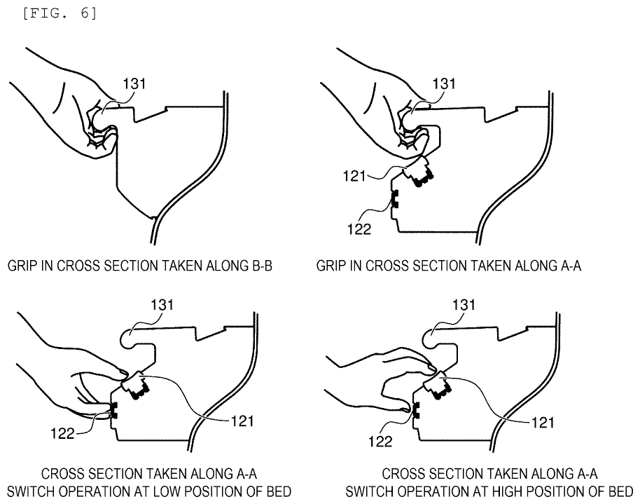

FIG. 6 is a view illustrating operations. As illustrated in FIG. 6, the operator can horizontally move and vertically move the first bed member 112 by gripping the grip portion 131. When the bed is at a low position, the operation unit 121 can be operated with a thumb while pressing the erroneous operation prevention button 122 with an index finger and the like. When the bed is at a high position, the operation unit 121 can be operated with the index or the like while pressing the erroneous operation prevention button 122 with the thumb. In this way, when an angle is provided between a placement surface (second predetermined angle surface) of the erroneous operation prevention button 122 and a placement surface (first predetermined angle surface) of the operation unit 121, simultaneous operation can be performed in a motion close to the gripping motion, and operability is improved.

As described above, the disclosed radiotherapy bed includes the base member 111, the first bed member 112 that is placed on the base member 111 and whose position with respect to the base member 111 can be mechanically controlled, and the second bed member 113 that is placed on the first bed member 112 and whose position with respect to the first bed member 112 can be mechanically controlled. The second bed member 113 is movable in a direction in which a treatment subject placed on the second bed member 113 is carried into an area where a radiation is to be emitted by a radiotherapy device and in a direction in which the treatment subject is carried out from the area where the radiation is to be emitted by the radiotherapy device, and the first bed member 112 has the grip portion 131 on a surface along the carrying-in direction of the treatment subject.

Therefore, when the operator manually adjusts the position of the bed, a work posture of the operator can be guided so as to grip the grip portion 131, and an inappropriate contact with the radiotherapy bed can be prevented. Therefore, a failure or a malfunction can be prevented.

In addition, in the disclosed radiotherapy bed, the first bed member 112 is movable in the horizontal direction and the vertical direction with respect to the floor, and the grip portion 131 has a shape in which the upper surface of the first bed member 112 protrudes in a direction perpendicular to the carrying-in direction and a recessed portion on which a finger of an operator is caught is provided below the protruding portion.

With such a shape suitable for gripping, the work posture of the operator can be reliably guided, and the workability can be improved.

Further, the first bed member 112 includes an operation unit 121 that receives an operation from the operator at a position where the grip portion 131 serves as an eave.

Therefore, the grip portion 131 prevents a liquid from directly coming into contact with the operation unit 121, so that a failure and a malfunction can be prevented.

The first bed member 112 includes a first predetermined angle surface having a first predetermined angle with respect to the upper surface of the first bed member 112, and the operation unit 121 is provided on the first predetermined angle surface.

Further, it is characterized in that the operation unit 121 is disposed such that the eave cannot reach an extension line in a direction perpendicular to the first predetermined angle surface from the operation unit 121.

Therefore, the visibility and operability of the operation unit 121 can be improved.

### The First Bed Member 112 Includes a Second

predetermined angle surface having a second predetermined angle different from the first predetermined angle with respect to the upper surface of the first bed member 112. The erroneous operation prevention button 122 is provided on the second predetermined angle surface, and the erroneous operation prevention button 122 and the operation unit 121 are provided close to each other. The operation unit 121 receives at least a part of the operations on condition that the erroneous operation prevention button 122 is being pressed.

As an example, the second predetermined angle surface is perpendicular to the floor.

In this way, by disposing the erroneous operation prevention button 122 and the operation unit 121 close to each other on different surfaces, erroneous operations can be reliably prevented while improving the operability.

The invention is not limited to the above-described embodiment and includes various modifications. For example, the above-described embodiment has been described in detail to facilitate understanding of the invention, and the invention is not necessarily limited to those including all the configurations described above. In addition to deletion of such a configuration, it is also possible to replace or add a configuration.

For example, in the above embodiment, the configuration in which the first bed member and the second bed member are combined to control the positional relationship of the three axial directions of X, Y, and Z, the pitch, the roll, and the like with respect to the area where radiation is to be emitted by the radiotherapy device has been exemplified, but it is not necessary to control all of these positional relationships. As an example, the radiotherapy device may be configured to perform position control in the Y direction.

### REFERENCE SIGNS LIST

100: radiotherapy bed
111: base member
112: first bed member
113: second bed member
121: operation unit
131: erroneous operation prevention button
131: grip portion

1. A radiotherapy bed comprising:
a base member;
a first bed member that is placed on the base member and whose position with respect to the base member is mechanically controllable; and
a second bed member that is placed on the first bed member and whose position with respect to the first bed member is mechanically controllable, wherein
the second bed member is movable in a direction in which a treatment subject placed on the second bed member is carried into an area where a radiation is to be emitted by a radiotherapy device and in a direction in which the treatment subject is carried out from the area where the radiation is to be emitted by the radiotherapy device, and
the first bed member has a grip portion on a surface along a carrying-in direction of the treatment subject.

2. The radiotherapy bed according to claim 1, wherein
the first bed member is movable in a horizontal direction and a vertical direction with respect to a floor, and
the grip portion has a shape in which an upper surface of the first bed member protrudes in a direction perpendicular to the carrying-in direction, and a recessed portion on which a finger of an operator is caught is provided below a protruding portion.

3. The radiotherapy bed according to claim 2, wherein
the first bed member includes an operation unit configured to receive an operation from the operator at a position where the grip portion serves as an eave.

4. The radiotherapy bed according to claim 3, wherein
the first bed member includes a first predetermined angle surface having a first predetermined angle with respect to the upper surface of the first bed member, and
the operation unit is provided on the first predetermined angle surface.

5. The radiotherapy bed according to claim 4, wherein
the operation unit is disposed such that the eave does not reach an extension line in a direction perpendicular to the first predetermined angle surface from the operation unit.

6. The radiotherapy bed according to claim 4, wherein
the first bed member includes a second predetermined angle surface having a second predetermined angle different from the first predetermined angle with respect to the upper surface of the first bed member,
an erroneous operation prevention button is provided on the second predetermined angle surface,
the erroneous operation prevention button and the operation unit are provided close to each other, and
the operation unit receives at least a part of operations on condition that the erroneous operation prevention button is being pressed.

7. The radiotherapy bed according to claim 6, wherein
the second predetermined angle surface is perpendicular to the floor.

8. A radiotherapy bed comprising:
a base member; and
a bed member that is placed on the base member and
whose position with respect to the base member is mechanically controllable, wherein
the bed member allows a positional relationship between a treatment subject placed on the bed member and an area where a radiation is to be emitted by a radiotherapy device to be controllable, and
the bed member has a grip portion on a surface along a height direction of the treatment subject.
